(19) United States
(12) Patent Application Publication
(10) Pub. No.: US 2010/0160755 A1
(43) Pub. Date: Jun. 24, 2010

(54) Polyelectrolytes as Sublayers on Electrochemical Sensors

(75) Inventors: Henry W. Oviatt, Temecula, CA (US); James R. Petisce, San Clemente, CA (US)

Correspondence Address: EDWARDS LIFESCIENCES CORPORATION, LEGAL DEPARTMENT, ONE EDWARDS WAY, IRVINE, CA, 92614, US

(73) Assignee: Edwards Lifesciences Corporation, Irvine, CA (US)

(21) Appl. No.: 12/632,623

(22) Filed: Dec. 7, 2009

Related U.S. Application Data
(60) Provisional application No. 61/140,826, filed on Dec. 24, 2008.

(51) Int. Cl. A61B 5/1473; G01N 27/26; C12Q 1/26; C12Q 1/00; C12Q 1/54

(52) U.S. Cl. 600/345; 204/421; 204/403.01; 204/403.14; 204/403.1; 204/406

(57) ABSTRACT

Disclosed herein is an electrochemical sensor for measuring an analyte in a subject. More particularly, sensors comprising a polyelectrolyte layer at least partially covering the electroactive surface of an electrode are disclosed.

### CROSS-REFERENCE TO RELATED APPLICATIONS

This application claims the benefit of U.S. Provisional Application No. 61/140,826, filed Dec. 24, 2008, which is hereby incorporated by reference herein in its entirety.

### FIELD OF THE INVENTION

The present invention relates generally to devices for measuring an analyte in a subject. More particularly, the present invention relates to devices for measurement of an analyte that incorporates a sensor comprising a polyelectrolyte layer for providing rapid and accurate analyte levels after deployment.

### BACKGROUND

In certain medical applications, patients in ICU or other emergency situations may often be fitted with invasive appliances such as catheters so that vital fluids or medicine may be administered intravenously. A physician determining an intravenous physiologic intervention for a patient needs to know vital physiological information as quickly as possible that may only be determined through blood chemistry analysis. Just how quickly the information is needed depends on the gravity of the situation. In some cases, the speed with which a physiological parameter may be determined may be the difference between life and death. In those situations, the practice of drawing a blood sample and sending it off for laboratory analysis may be entirely too slow.

Among many problems impeding the development of a practical rapid and accurate amperometric sensor is a current need for the sensor technology to “break-in” or otherwise rapidly achieve chemical, electrical and physical equilibrium with its environment and provide a signal that is an accurately representative of the true analyte level. Attempts to reduce break-in in amperometric sensors have been addressed in a number of ways, for example by using separate and distinct hydrophilic layers in a multi-membrane-based system or by incorporating an aqueous reservoir or environment about the sensor, albeit with limited success because the break-in improvements to date for these systems have generally only provided limited improvements in break-in. In certain cases, such as an intensive care unit (ICU) setting or for continuous glucose monitoring (CGM) applications, break-in requirements would ideally be a few minutes or seconds. Current amperometric sensors available on the market may not be capable of achieving the required rapid break-in performance needed for specific applications, such as ICU monitoring of analyte levels in a subject, particularly but not limited to blood glucose levels.

Additionally, it is generally known that in some circumstances, a separate “hydrophilic domain” may be employed in a sensor between the electroactive surface(s) (e.g., working and/or reference electrodes) and additional layers, such as an interference layer or an enzyme layer, to create stable sensor activity. Electrolytes are commonly utilized in these hydrophilic domains to aid electron transfer at the electroactive surface of an electrode and between redox centers of the enzyme layer. Traditional “electrolyte phases” have contained low molecular weight ionic components, such as low molecular weight salts. These low molecular weight salts, or “fugitive species,” often diffuse from the sensor during use. Diffusion of fugitive species may, for example, alter basic sensor properties.

There exists an unmet need to provide intravenous amperometric sensing, in which the concentration of a analyte present in a patient's bloodstream may be determined by locating, within the circulatory system, a sensor comprising an enzyme electrode that produces a rapid and accurate electrical current proportional to the true analyte concentration.

### SUMMARY

In general, electrochemical analyte sensors and sensor assemblies are disclosed that provide rapid chemical, electrical, and physical equilibrium with their environment and as a result, provide fast and accurate analyte levels. Such sensors are of particular use in more demanding sensing applications, such as ICU monitoring. The invention disclosed herein relates to sensors and sensor assemblies that incorporate high molecular weight moieties having pendent ionizable groups that are at least partially in contact with the electroactive sensor.

In one aspect, an electrochemical analyte sensor is provided. The sensor comprises at least one non-reference electrode having an electroactive surface, and a polyelectrolyte layer in contact with at least a portion of the electroactive surface of the at least one non-reference electrode.

In another aspect, an electrochemical analyte sensor is provided comprising at least one non-reference electrode having an electroactive surface, a polyelectrolyte layer in contact with at least part of the electroactive surface of the at least one non-reference electrode, and an enzyme layer. The enzyme layer is in contact with the polyelectrolyte layer and covers the polyelectrolyte layer.

In another aspect, an electrochemical analyte sensor is provided comprising at least one conductive ink electrode that has an electroactive surface and a polyelectrolyte layer in contact with a portion of the electroactive surface of the conductive ink electrode. An interference layer covers the polyelectrolyte layer. An enzyme layer covers the interference layer and the polyelectrolyte layer. A polymer membrane covers the enzyme layer, the interference layer, the polyelectrolyte layer.

In one aspect, an electrochemical analyte sensor assembly is provided. The assembly comprises a flex circuit comprising at least one reference electrode and at least one working electrode, the at least one working electrode having an electroactive surface capable of providing a detectable electrical output upon interaction with an electrochemically detectable species. A polyelectrolyte layer at least partially contacts the electroactive surface. The flex circuit is electrically configurable to a control unit capable of at least receiving the detectable electrical output.

In another aspect, an electrochemical analyte sensor assembly is provided comprising a flex circuit comprising at least one reference electrode and at least one working electrode, the at least one working electrode having an electroactive surface capable of providing a detectable electrical output upon interaction with an electrochemically detectable species. A polyelectrolyte layer comprising sulfonate functionality is in contact with at least a portion of the electroactive surface. An interference layer comprising cellulose acetate or cellulose acetate butyrate or a combination thereof contacts the polyelectrolyte layer. An enzyme layer comprising glucose oxidase is in contact with the interference layer. A second polymer membrane covers the enzyme layer, the interference layer, the polyelectrolyte layer, and at least a portion of the electroactive surface. The flex circuit can be electrically coupled to a control unit capable of at least receiving the detectable electrical output.

In another aspect, a method of measuring an analyte in a subject is provided. The method comprises providing a catheter comprising one of the sensor assemblies as described herein, contacting bodily fluids of a subject, and measuring an analyte.

### BRIEF DESCRIPTION OF THE DRAWINGS

FIG. 1 shows an amperometric sensor in the form of a flex circuit having a working electrode according to an embodiment of the invention.

FIG. 2 is a side cross-sectional view of a working electrode portion of the sensor of shown prior to application of a membrane according to an embodiment of the invention.

FIG. 3 is a cross-sectional view of the working electrode portion of the sensor as in FIG. 2, shown after application of the membrane according to an embodiment of the invention.

FIG. 4 is a side view of a multi-lumen catheter with a sensor assembly according to an embodiment of the invention.

FIG. 5 is a detail of the distal end of the multi-lumen catheter of FIG. 4 according to an embodiment of the invention.

FIGS. 6 and 6A are graphs depicting net current verses step changes in glucose concentration for a sensor embodiment comprising polystyrene sulfonate-co-maleic acid polyelectrolyte.

FIGS. 7 and 7A are linear correlation graphs depicting net current verses glucose concentration for a sensor embodiment comprising polystyrene sulfonate-co-maleic acid polyelectrolyte.

FIGS. 8 and 8A are graphs depicting net current output verses glucose concentration for a sensor embodiment comprising benzalkonium heparin polyelectrolyte.

FIGS. 9 and 9A are linear correlation graphs depicting net current verses glucose concentration for a sensor embodiment comprising benzalkonium heparin polyelectrolyte.

### DETAILED DESCRIPTION

Conventional glucose sensor technology typically relies on the use of a platinum based electrode surface that oxidizes hydrogen peroxide produced from the reaction between glucose oxidase and glucose. In order to provide for stable sensor activity, low molecular weight salts have been used at the interface of the electrode to provide an ionic media at the sensor interface. Undesirably, these low molecular weight, ionic components may diffuse out (fugative) of the sensor during use which may result in a change of the basic properties of the sensor.

Disclosed and described herein are polyelectrolyte compounds that have a high molecular weight (greater than a few thousand, up to millions of Daltons) that would be restricted or inhibited from diffusing away (non-fugative) from the electrode surface through the various membranes of the sensor. The molecular weight of the polyelectrolyte is such that fugitive ionic species are prevented or substantially inhibited from leaving the sensor electrode environment and more particularly, fugitive species are prevented or substantially inhibited from leaving the sensor's electrode environment when the sensor is initially deployed. By restricting and/or inhibiting diffusion away of the polyelectrolyte from the sensor electrode, an osmotic gradient may be provided for the sensor when first hydrated which would facilitate rapid equilibrium of sensor to a hydrated state. Such rapid hydration may reduce the “run-in” time associated with the sensor. The sensor comprising a polyelectrolyte would also allow for multiple hydration and dehydration cycles without the loss of ionic species during the initial hydration, allowing pre-testing of the sensor before final packaging, for example. The polyelectrolytes can be either cationic or anionic, and natural or synthetic, with the polymer chain being the support for a sequence of anions or cations. The polyelectrolyte may further provide a buffer system for a desirable pH range in proximity to the electrode and/or enzyme and/or may provide electroneutrality to the sensor electrode environment. The polyelectrolyte may be positioned anywhere within the membrane architecture of the sensor, for example, anywhere under the outermost membrane of the sensor.

The following description and examples illustrate some exemplary embodiments of the disclosed invention in detail. Those of skill in the art will recognize that there may be numerous variations and modifications of this invention that may be encompassed by its scope. Accordingly, the description of a certain exemplary embodiment is not intended to limit the scope of the present invention.

### DEFINITIONS

In order to facilitate an understanding of the various aspects of the invention, the following are defined below.

The term “analyte” as used herein refers without limitation to a substance or chemical constituent of interest in a biological fluid (for example, blood) that may be analyzed. The analyte may be naturally present in the biological fluid, the analyte may be introduced into the body, or the analyte may be a metabolic product of a substance of interest or an enzymatically produced chemical reactant or chemical product of a substance of interest. Preferably, analytes include but are not limited to chemical entities capable of reacting with at least one enzyme and quantitatively yielding an electrochemically reactive product that is either amperiometrically or voltammetrically detectable.

The phrases and terms “analyte measuring device,” “sensor,” and “sensor assembly” as used herein refer without limitation to an area of an analyte-monitoring device that enables the detection of at least one analyte. For example, the sensor may comprise a non-conductive portion, at least one working electrode, a reference electrode, and a counter electrode (optional), forming an electrochemically reactive surface at one location on the non-conductive portion and an electronic connection at another location on the non-conductive portion, and a one or more layers over the electrochemically reactive surface.

The phrase “cellulose acetate butyrate” as used herein refers without limitation to compounds obtained by contacting cellulose with acetic anhydride and butyric anhydride.

The term “comprising” and its grammatical equivalents, as used herein is synonymous with “including,” “containing,” or “characterized by,” and is inclusive or open-ended and does not exclude additional, unrecited elements or method steps.

The phrases “continuous analyte sensing” and “continual analyte sensing” (and the grammatical equivalents “continuously” and continually”) as used herein refer without limitation to a period of analyte concentration monitoring that is continuously, continually, and/or intermittently (but regularly) performed.

The phrase “continuous glucose sensing” as used herein refers without limitation to a period of glucose concentration monitoring that is continuously, continually, and/or intermittently (but regularly) performed. The period may, for example, at time intervals ranging from fractions of a second up to, for example, 1, 2, or 5 minutes, or longer.

The term “cover” and its grammatical equivalents is used herein refers without limitation to its normal dictionary definition. The term cover is inclusive of one or more intervening layers. For example, a layer covering at least a portion of an electroactive surface is inclusive of one or more intervening layers between the layer and the electroactive surface.

The terms “crosslink” and “crosslinking” as used herein refer without limitation to joining (e.g., adjacent chains of a polymer and/or protein) by creating covalent or ionic bonds. Crosslinking may be accomplished by known techniques, for example, thermal reaction, chemical reaction or ionizing radiation (for example, electron beam radiation, UV radiation, X-ray, or gamma radiation). For example, reaction of a dialdehyde such as glutaraldehyde with a hydrophilic polymer-enzyme composition would result in chemical crosslinking of the enzyme and/or hydrophilic polymer.

The phrase “break-in” as used herein refers without limitation to a time duration, after initial sensor use, where an electrical output from the sensor achieves a substantially constant value following an electrical input to the sensor. For example, following a potential input to the sensor, an immediate break-in would be a substantially constant current output from the sensor. By way of example, an immediate break-in for a glucose electrochemical sensor after a potential input, would be a current output representative of +/−5 mg/dL or less of a calibrated glucose concentration within about two minutes or less after deployment. Other methods of eliminating or reducing the break-in time of the sensor may be used in combination with the embodiments disclosed herein, such as, but not limited to, configuring the sensor electronics by applying different voltage settings, starting with a higher voltage setting and then reducing the voltage setting and/or pre-treating the operating electrode with a negative electric current at a constant current density. Break-in is inclusive of chemical/electrical equilibrium of one or more of the sensor components such as membranes, layers, enzymes and electronics, and may occur prior to calibration of the sensor output. The phrase “break-in” is well documented and is appreciated by one skilled in the art of electrochemical glucose sensors, however it may be exemplified for a glucose sensor, as the time at which reference glucose data (e.g., from an self monitoring of blood glucose (SMBG) meter) is within +/−5 mg/dL of the measured glucose sensor data.

The phrase “electroactive surface” as used herein is refers without limitation to a surface of an electrode where an electrochemical reaction takes place. The electroactive surface includes the surface of any of one or more working electrodes (WE), any of one or more reference electrodes (RE), any one or more blank electrodes (BE), and any of one or more counter electrodes (CE). For example, at a predetermined potential, H₂O₂ reacts with the electroactive surface of a working electrode to produce two protons (2H+), two electrons (2e⁻) and one molecule of oxygen (O₂), for which the electrons produce a detectable electronic current. The electroactive surface may include on at least a portion thereof, a chemically or covalently bonded adhesion promoting agents such as aminoalkylsilanes and the like.

The term “subject” as used herein refers without limitation to mammals, particularly humans and domesticated animals.

The terms “interferants,” “interferents” and “interfering species,” as used herein refer without limitation to effects and/or species that otherwise interfere with a measurement of an analyte of interest in a sensor to produce a signal that does not accurately represent the analyte measurement. For example, in an electrochemical sensor, interfering species may be compounds with oxidation or reduction potentials that substantially overlap the oxidation potential of the analyte to be measured.

The phrase “enzyme layer” as used herein refers without limitation to a permeable or semi-permeable layer comprising an enzyme contained within one or more domains that may be permeable to reactants and/or co-reactants employed in determining the analyte of interest. As an example, an enzyme layer comprises an immobilized glucose oxidase enzyme in a hydrophilic polymer, which catalyzes an electrochemical reaction with glucose and oxygen to permit measurement of a concentration of glucose.

The phrase “flux-limiting membrane” refers to a semi-permeable layer that controls the flux of at least one analyte to the underlying enzyme layer. By way of example, for a glucose sensor, the membrane preferably renders oxygen in a non-rate-limiting excess. As a result, the upper limit of linearity of glucose measurement is extended to a much higher value than that which is achieved without the flux-limiting membrane.

The term “polyelectrolyte” as used herein refers to a high molecular weight material having pendent ionizable groups. The molecular weight of polyelectrolytes may range from a few thousand to millions of Daltons. In one aspect, polyelectrolytes are exclusive of polymers with terminal ionizable groups and essentially no pendent ionizable groups, for example, Nafion.

### Sensor System and Sensor Assembly

The aspects of the invention herein disclosed relate to the use of an analyte sensor system that measures a concentration of analyte of interest or a substance indicative of the concentration or presence of the analyte. The sensor system is a continuous device, and may be used, for example, as or part of a subcutaneous, transdermal (e.g., transcutaneous), or intravascular device. The analyte sensor may use an enzymatic, chemical, electrochemical, or combination of such methods for analyte-sensing. The output signal is typically a raw signal that is used to provide a useful value of the analyte of interest to a user, such as a patient or physician, who may be using the device. Accordingly, appropriate smoothing, calibration, and evaluation methods may be applied to the raw signal.

One exemplary aspect of the sensor comprises at least a portion of the exposed electroactive surface of a working electrode surrounded by a plurality of layers. A polyelectrolyte layer is deposited over and in contact with at least a portion of the electroactive surfaces of the sensor to create a more stable electrochemical environment. An interference layer may optionally be deposited over and in contact with at least a portion of the polyelectrolyte layer to provide protection from the biological environment and/or limit or block interferents. An enzyme layer is deposited over and in contact with at least a portion of the interference layer or polyelectrolyte layer. A membrane may at least partially cover the enzyme layer, the interference layer, the polyelectrolyte layer, and the electroactive surface.

One exemplary embodiment described in detail below utilizes a medical device, such as a catheter, with an analyte sensor assembly. In one aspect, a medical device with a glucose sensor assembly is provided for inserting the sensor into a subject's vascular system. The medical device with the analyte sensor assembly may include associated therewith an electronics unit associated with the sensor, and a receiver for receiving and/or processing sensor data. Although a few exemplary embodiments of continuous glucose sensors may be illustrated and described herein, it should be understood that the disclosed embodiments may be applicable to any device capable of substantially continual or substantially continuous measurement of a concentration of an analyte of interest and for providing an rapid and accurate output signal that is representative of the concentration of that analyte.

### Electrode and Electroactive Surface

The electrode and/or the electroactive surface of the sensor or sensor assembly disclosed herein comprises a conductive material, such as platinum, platinum-iridium, palladium, graphite, gold, carbon, conductive polymer, alloys, ink, or the like. Although the electrodes may be formed by a variety of manufacturing techniques (bulk metal processing, deposition of metal onto a substrate, or the like), it may be advantageous to faun the electrodes from screen printing techniques using conductive and/or catalyzed inks. The conductive inks may be catalyzed with noble metals such as platinum and/or palladium.

In one aspect, the electrodes and/or the electroactive surfaces of the sensor or sensor assembly are formed on a flexible substrate. In one aspect, the electrodes and/or the electroactive surfaces of the sensor or sensor assembly are formed on a flexible substrate that is a flex circuit. In one aspect, a flex circuit is part of the sensor and comprises a substrate, conductive traces, and electrodes. The traces and electrodes may be masked and imaged onto the substrate, for example, using screen printing or ink deposition techniques. The trace and the electrodes, and the electroactive surface of the electrode may be comprised of a conductive material, such as platinum, platinum-iridium, palladium, graphite, gold, carbon, silver, conductive polymer, alloys, ink or the like.

In one aspect, a counter electrode is provided to balance the current generated by the species being measured at the working electrode. In the case of a glucose oxidase based glucose sensor, the species being measured at the working electrode is H₂O₂. Glucose oxidase catalyzes the conversion of oxygen and glucose to hydrogen peroxide and gluconate according to the following reaction: Glucose+O₂→Gluconic acid+H₂O₂. Oxidation of H₂O₂ by the working electrode is balanced by reduction of any oxygen present, enzyme generated H₂O₂, or other reducible species at the counter electrode. The H₂O₂ produced from the glucose oxidase reaction reacts at the surface of the working electrode and produces two protons (2H⁺), two electrons (2e⁻), and one oxygen molecule (O₂).

In one aspect, additional electrodes may be included within the sensor or sensor assembly, for example, a three-electrode system (working, reference, blank and counter electrodes) and/or one or more additional working electrodes configured as a baseline subtracting electrode, or which is configured for measuring additional analytes. The two working electrodes may be positioned in close proximity to each other, and in close proximity to the reference electrode. For example, a multiple electrode system may be configured wherein a first working electrode is configured to measure a first signal comprising glucose and baseline and an additional working electrode substantially similar to the first working electrode without an enzyme disposed thereon is configured to measure a baseline signal consisting of baseline only. In this way, the baseline signal generated by the additional electrode may be subtracted from the signal of the first working electrode to produce a glucose-only signal substantially free of baseline fluctuations and/or electrochemically active interfering species.

In one aspect, the sensor comprises from 2 to 4 electrodes. The electrodes may include, for example, the counter electrode (CE), working electrode (WE1), reference electrode (RE), and optionally a second working electrode (WE2). In one aspect, the sensor will have at least a CE and WE1. In one aspect, the addition of a WE2 is used, which may further improve the accuracy of the sensor measurement.

The electroactive surface of the electrodes (WE, CE, BE and RE) may be treated prior to application of any subsequent layers, including the layer described herein. Surface treatments may include for example, chemical, plasma or laser treatment of at least a portion of the electroactive surface. The electrodes may be chemically or covalently contacted with one or more adhesion promoting agents. Adhesion promoting agents may include for example, aminoalkylalkoxylsilanes, epoxyalkylalkoxylsilanes and the like. For examples, one or more of the electrodes may be chemically or covalently contacted with a solution containing 3-glycidoxypropyltrimethoxysilane.

In some alternative embodiments, the exposed surface area of the working (and/or other) electrode may be increased by altering the cross-section of the electrode itself. Increasing the surface area of the working electrode may be advantageous in providing an increased signal responsive to the analyte concentration, which in turn may be helpful in improving the signal-to-noise ratio, for example. The cross-section of the working electrode may be defined by any regular or irregular, circular or non-circular configuration.

### Polyelectrolyte Layer

Polyelectrolytes are high molecular weight materials having pendent ionizable groups. As electrolytes, polyelectrolytes exhibit the advantageous ionic properties required for stable sensor functioning, such as charge neutralization and charge transfer abilities. Due to their large size, polyelectrolytes substantially reduce or eliminate diffusion of the electrolytic species through the sensor membranes. While not being held to any particular theory, it is believed that the polyelectrolyte layer creates an osmotic gradient upon hydration, which facilitates rapid equilibrium and leads to a reduction in a sensor's “break-in” time, while also allowing the top sensor membrane to be hydrated and dehydrated multiple times without substantial diffusion of fugitive species. Thus, a polyelectrolyte layer as described herein would substantially maintain electroneutrality while providing a non-fugitive buffering system.

Different aspects of the invention may comprise different polyelectrolytes. For example, some aspects of the invention may comprise a polyelectrolyte layer comprised of polyacids, while other aspects may utilize polybases or polyampholytes in the polyelectrolyte layer. Further aspects may utilize a polyelectrolyte layer comprising a polyelectrolyte salt, or polysalt.

Some aspects of the invention may utilize a polyelectrolyte layer comprising pharmaceutically acceptable polysalts. A pharmaceutically acceptable salt is one which is safe and effective for use in humans. For example, pharmaceutically acceptable salts may include polycations with counterions comprising sulfate, pyrosulfate, bisulfate, sulfite, bisulfite, phosphate, monohydrogenphosphate, dihydrogenphosphate, metaphosphate, pyrophosphate, (bi)carbonate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, beta-hydroxybutyrate, glycolate, maleate, tartrate, methanesulfonate, propanesulfonate, naphthalene-1-sulfonate, naphthalene-2-sulfonate, mandelate, or polyanions with positive counterions from elements such as aluminum, calcium, lithium, magnesium, potassium, sodium, and zinc, or from organic compounds such as benzalkonium, pyridinium, quaternary alkyl or arylammonium, or other organic cations, among others. One skilled in the art of polymer science can appreciate the very wide diversity of possible combinations of polyions (polymers containing repeat linkages with positive or negative charges) and the associated counterions, and will recognize that the list above is not by any means exhaustive, and other possible combinations are considered to be inclusive, including the possible combination of one or more polyanion and one or more polycation to form a relatively insoluble polyelectrolyte layer.

Generally, polyelectrolytes have numerous ionizable groups, and thus may be highly charged. In one aspect, the polyelectrolyte layer may be comprised of polyelectrolytes with multiple ionizable groups. In a further aspect, the polyelectrolyte layer may be comprised of highly charged polyelectrolytes without terminal ionizable groups.

In one aspect, the polyelectrolyte layer may be comprised of a polyelectrolyte comprising sulfonate functionality. Incorporating a polyelectrolyte with sulfonate functionality may be advantageous for analyte sensors, as sulfonate groups are the salts of strong acids and therefore have little influence on the local pH. For example, a polystyrene sulfonate, such as poly(sodium-4-styrene sulfonate), or copolymers of polystyrene sulfonate and maleic acid, such as poly(4-styrene sulfonic acid-co-maleic acid)Na salt, or blends or copolymers thereof may be utilized.

In a further aspect, the polyelectrolyte layer may be comprised of heparin. Heparin, a naturally occurring polysaccharide polyelectrolyte with sulfonate functionality, has numerous advantages for use in medical devices, including sensors. In one aspect, benzalkonium heparin is used as the polyelectrolyte. Other salts of heparin may be used, preferably pharmaceutically acceptable salts of heparin. Benzalkonium heparin is frequently used as an anticoagulant on medical devices or used to inhibit blood coagulation in a patient. Thus, one advantage of heparin polyelectrolytes, such as benzalkonium heparin, is that in the event of an outer membrane failure of the sensor, heparin polyelectrolyte layer being released from the sensor would likely not cause a toxic response in the subject. An additional advantage of heparin solutions, being prepared in alcohol solvents, is their potential to enhance the wettability of a hydrophobic electrode surface during their application to the sensor electrodes.

In another aspect, the polyelectrolyte layer may comprise carboxylic acid functionality. Carboxylic acids may be advantageous in sensors as they may act as buffers under the electrode, thus keeping the system in a desirable pH range under brief local pH changes. Examples of suitable polyelectrolytes with carboxylic acid functionality include polyacrylic acid and polyalkylacrylic acid, where the alkyl is C₁-C₄. In one aspect, the polyelectrolytes with carboxylic acid functionality include polyacrylic acid, polymethacrylic and copolymers or blends thereof.

It will be obvious to one of ordinary skill in the art that any reasonable polyelectrolyte salt could be utilized in this invention. The polyelectrolyte salts discussed above are offered merely by way of example, and are not meant to limit the instant invention.

### Additional Layers

The electroactive surface of the electrodes may be coated with a material selected from cellulose ester derivatives, silicones, polytetrafluoroethylene, polyethylene-co-tetrafluoroethylene, polyolefin, polyester, polycarbonate, biostable polytetrafluoroethylene, homopolymers, copolymers, terpolymers or blends of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), polyurethanes, cellulosic polymers, polysulfones, tetrafluoroethylene-perfluoro-3,6-dioxa-4-methyl-7-octenesulfonic acid copolymer (Nation) and block copolymers thereof including, for example, di-block, tri-block, alternating, random and graft copolymers. Blends of the above polymers may be used.

In one preferred aspect, electrodes may be coated with a material layer is an interferant layer, such that the layer is effective at reducing or eliminating diffusion of interfering species relative to, for example, hydrogen peroxide. Interferents may be molecules or other species that may be reduced or oxidized at the electrochemically reactive surfaces of the sensor, either directly or via an electron transfer agent, to produce a false positive analyte signal (e.g., a non-analyte-related signal), or may be exogenous or endogenous compounds that inhibit the ability of the electroactive metals in the electrode surface from functioning efficiently, reducing the overall electrochemical signal. This false positive signal generally causes the subject's analyte concentration to appear higher than the true analyte concentration. For example, in a hypoglycemic situation, where the subject has ingested an interferent (e.g., acetaminophen), the artificially high glucose signal may lead the subject or health care provider to believe that they are euglycemic or, in some cases, hyperglycemic. As a result, the subject or health care provider may make inappropriate or incorrect treatment decisions.

In one aspect, an interference layer is provided on the sensor or sensor assembly that substantially restricts or eliminates the passage through of one or more interfering species. Interfering species for a glucose sensor include, for example, acetaminophen, ascorbic acid, bilirubin, cholesterol, creatinine, dopamine, ephedrine, ibuprofen, L-dopa, methyl dopa, salicylate, tetracycline, tolazamide, tolbutamide, triglycerides, urea, and uric acid, or other electroactive or inhibitory substances. The interference layer may be less permeable to one or more of the interfering species than to a target analyte species.

In an embodiment, the interference layer is formed from one or more cellulosic derivatives. In one aspect, mixed ester cellulosic derivatives may be used, for example cellulose acetate butyrate, cellulose acetate phthalate, cellulose acetate propionate, cellulose acetate trimellitate, as well as their copolymers and terpolymers, with other cellulosic or non-cellulosic monomers, including cross-linked variations of the above. Other polymers, such as polymeric polysaccharides having similar properties to cellulosic derivatives, may be used as an interference material or in combination with the above cellulosic derivatives. Other esters of cellulose may be blended with the mixed ester cellulosic derivatives.

In one aspect, the interference layer is formed from cellulose acetate butyrate. Cellulose acetate butyrate is a cellulosic polymer having both acetyl and butyl groups, and may also include hydroxyl groups. A cellulose acetate butyrate having about 35% or less acetyl groups, about 10% to about 25% butylryl groups, and hydroxyl groups making up the remainder may be used. A cellulose acetate butyrate having from about 25% to about 34% acetyl groups and from about 15 to about 20% butylryl groups may also be used. However, other amounts of acetyl and butylryl groups may be used. A preferred cellulose acetate butyrate contains from about 28% to about 30% acetyl groups and from about 16% to about 18% butylryl groups.

Cellulose acetate butyrate with a molecular weight of about 10,000 daltons to about 75,000 daltons is preferred, preferably from about 15,000, 20,000, or 25,000 daltons to about 50,000, 55,000, 60,000, 65,000, or 70,000 daltons, and more preferably about 65,000 daltons is employed. In certain embodiments, however, higher or lower molecular weights may be used or a blend of two or more cellulose acetate butyrates having different molecular weights may be used.

A plurality of layers of cellulose acetate butyrate may be combined to form the interference layer in some embodiments. For example, two or more layers may be employed. It may be desirable to employ a mixture of cellulose acetate butyrates with different molecular weights in a single solution, or to deposit multiple layers of cellulose acetate butyrate from different solutions comprising cellulose acetate butyrate of different molecular weights, different concentrations, and/or different chemistries (e.g., wt % functional groups). Additional substances in the casting solutions or dispersions may be used, e.g., casting aids, defoamers, surface tension modifiers, functionalizing agents, crosslinking agents, other polymeric substances, substances capable of modifying the hydrophilicity/hydrophobicity of the resulting layer, and the like. Nonetheless, in one aspect, the interference layer is substantially free of polyelectrolytes.

The precursor composition of the layer may be sprayed, cast, deposited, or dipped directly to the electroactive surface(s) of the electrodes. The dispensing of the precursor composition of the layer may be performed using any known thin film technique. Two, three or more layers of precursor composition of the layer may be formed by the sequential application and curing and/or drying.

In one aspect, the concentration of solids in the casting solution may be adjusted to deposit a sufficient amount of solids or film on the electrode in one layer (e.g., in one dip or spray) to form a layer sufficient to block an interferent with an oxidation or reduction potential otherwise overlapping that of a measured species (e.g., H₂O₂)), measured by the sensor. For example, the casting solution's percentage of solids may be adjusted such that only a single layer is required to deposit a sufficient amount to form a functional interference layer that substantially prevents or reduces the equivalent glucose signal of the interferant measured by the sensor. A sufficient amount of intereference material would be an amount that substantially prevents or reduces the equivalent glucose signal of the interferant of less than about 30, 20, or 10 mg/dL. By way of example, the interference layer is preferably configured to substantially block about 30 mg/dL of an equivalent glucose signal response that otherwise would be produced by acetaminophen by a sensor without an interference layer. Such equivalent glucose signal response produced by acetaminophen would include a therapeutic dose of acetaminophen. Any number of coatings or layers formed in any order may be suitable for forming the interference layer of the embodiments disclosed herein.

The interference layer may be applied to provide a thickness of from about 0.05 micron or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns and more preferably still from about 1, 1.5 or 2 microns to about 2.5 or 3 microns. Thicker membranes may also be desirable in certain embodiments, but thinner membranes may be generally preferred because they generally have a lower affect on the rate of diffusion of hydrogen peroxide from the enzyme membrane to the electrodes.

In one aspect, polymers, such as Nafion®, may be used alone or in combination with a cellulosic derivative to provide the layer for the electroactive surface of the non-working electrode. For example, a layer of a 5 wt. % Nafion® casting solution was applied over a previously applied (e.g., and cured) layer of 8 wt. % cellulose acetate, e.g., by dip coating at least one layer of cellulose acetate and subsequently dip coating at least one layer Nafion® onto the electroactive surface of the non-working electrode. Any number of coatings or layers formed in any order may be suitable for forming the layer on the electroactive surface of the non-working electrode.

In other aspects, other polymer types may be utilized as a base material for the layer on the electroactive surface of the non-working electrode. For example, polyurethanes, polymers having pendant ionic groups, and polymers having controlled pore size, for example. By way of example, the layer on the non-working electrode may include a thin, hydrophobic membrane that is substantially non-swellable and restricts diffusion of high molecular weight species, such as biological components.

### Enzyme Layer

The sensor or sensor assembly disclosed herein generally includes an enzyme layer comprising an enzyme composition. In one aspect, the enzyme layer comprises a enzyme and a hydrophilic polymer. The hydrophilic polymer may be selected from poly-N-vinylpyrrolidone, poly-N-vinyl-3-ethyl-2-pyrrolidone, poly-N-vinyl-4,5-dimethyl-2-pyrrolidone, polyacrylamide, poly-N,N-dimethylacrylamide, polyvinyl alcohol, polymers with pendent ionizable groups and copolymers or blends thereof. Preferably, the enzyme layer comprises poly-N-vinylpyrrolidone. In one aspect, the enzyme layer comprises glucose oxidase, poly-N-vinylpyrrolidone and an amount of crosslinking agent sufficient to immobilize the enzyme and/or the poly-N-vinylpyrrolidone.

Most importantly, the molecular weight of the hydrophilic polymer of the enzyme layer is such that fugitive species are prevented or substantially inhibited from leaving the sensor environment and more particularly, fugitive species are prevented or substantially inhibited from leaving the enzyme's environment when the sensor is initially put into use.

The hydrophilic polymer-enzyme composition of the enzyme layer may further include at least one protein and/or natural or synthetic material. For example, the hydrophilic polymer-enzyme composition of the enzyme layer may further include serum albumins, polyallylamines, polyamines and the like, as well as combination thereof.

The enzyme layer composition may further include at least one polyelectrolyte as described above. For example, the enzyme layer composition may further include heparin, substituted polystyrenes, polycarboxylic acid, or pharmaceutically acceptable salts and combinations thereof. One of ordinary skill in the art should appreciate that other polyelectrolytes may also be utilized, for example, polyelectrolytes inclusive of those previously described.

The enzyme is preferably encapsulated within the hydrophilic polymer and may be cross-linked or otherwise immobilized therein. The enzyme may be cross-linked or otherwise immobilized optionally together with at least one protein and/or natural or synthetic material. In one aspect, the hydrophilic polymer-enzyme composition comprises glucose oxidase, bovine serum albumin, and poly-N-vinylpyrrolidone. The composition may further include a cross-linking agent, for example, a dialdehyde such as glutaraldehdye, to cross-link or otherwise immobilize the components of the composition.

In one aspect, other proteins or natural or synthetic materials may be substantially excluded from the hydrophilic polymer-enzyme composition of the enzyme layer. For example, the hydrophilic polymer-enzyme composition may be substantially free of bovine serum albumin. Bovine albumin-free compositions may be desirable for meeting various governmental regulatory requirements. Thus, in one aspect, the hydrophilic polymer-enzyme composition of the enzyme layer consists essentially of glucose oxidase, poly-N-vinylpyrrolidone and a cross-linking agent, for example, a dialdehyde such as glutaraldehdye, to cross-link or otherwise immobilize the components of the composition.

In one aspect, the enzyme composition comprises glucose oxidase, bovine serum albumin, and poly-N-vinylpyrrolidone. The composition may further include a cross-linking agent, for example, a dialdehyde such as glutaraldehdye, to cross-link or otherwise immobilize the components of the composition. In one aspect, the enzyme is encapsulated within a hydrophilic polymer and may be cross-linked or otherwise immobilized therein.

The enzyme layer thickness may be from about 0.05 microns or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns. Preferably, the enzyme domain is deposited by spray or dip coating, however, other methods of forming the enzyme layer may be used. The enzyme layer may be formed by dip coating and/or spray coating one or more layers at a predetermined concentration of the coating solution, insertion rate, dwell time, withdrawal rate, and/or desired thickness.

### Flux-Limiting Membrane

The sensor or sensor assembly may further include a membrane disposed over the subsequent layers described above. The membrane may function as a flux-limiting membrane. Although the following description is directed to a flux-limiting membrane for a glucose sensor, the flux-limiting membrane may be modified for other analytes and co-reactants as well. In one aspect, the sensor or sensor assembly includes a flux-limiting membrane disposed on the layer as herein disclosed.

The flux-limiting membrane comprises a semi-permeable material that controls the flux of oxygen and glucose to the underlying enzyme layer, preferably providing oxygen in a non-rate-limiting excess. As a result, the upper limit of linearity of glucose measurement is extended to a much higher value than that which is achieved without the flux-limiting membrane. In one embodiment, the flux-limiting membrane exhibits an oxygen to glucose permeability ratio of from about 50:1 or less to about 400:1 or more, preferably about 200:1.

In one aspect, the material that comprises the flux-limiting membrane may be a vinyl polymer appropriate for use in sensor devices as having sufficient permeability to allow relevant compounds to pass through it, for example, to allow an oxygen molecule to pass through in order to reach the active enzyme or electrochemical electrodes. Examples of materials which may be used to make flux-limiting membranes include vinyl polymers having vinyl acetate monomeric units. In a preferred embodiment, the flux-limiting membrane comprises poly ethylene vinylacetate (EVA polymer). In other aspects, the flux-limiting membrane comprises poly(methylmethacrylate-co-butyl methacrylate) blended with the EVA polymer. The EVA polymer or its blends may be cross-linked, for example, with diglycidyl ether.

In one aspect of the invention, the flux-limiting membrane excludes condensation polymers such as silicone and urethane polymers and/or copolymers or blends thereof. Such excluded condensation polymers typically contain residual heavy metal catalytic material that may otherwise be toxic if leached and/or difficult to completely remove, thus rendering their use in such sensors undesirable for safety and/or cost.

The EVA polymer may be provided from a source having a composition anywhere from about 9 wt % vinyl acetate (EVA-9) to about 40 wt % vinyl acetate (EVA-40). The EVA polymer is preferably dissolved in a solvent for dispensing on the sensor or sensor assembly. The solvent should be chosen for its ability to dissolve EVA polymer, to promote adhesion to the sensor substrate and enzyme electrode, and to form a solution that may be effectively applied (e.g. sprayed, or dip coated, or deposited by volume on the sensor element). Solvents such as cyclohexanone, xylene and isomers thereof, and tetrahydrofuran may be suitable for this purpose, but any solvent with sufficient chemical properties as to be a solvent for the EVA polymer would be suitable and could be determined by one skilled in the art. For an EVA material with a 40% vinyl acetate content, the solution may include about 0.5 wt % to about 15 wt % of the EVA polymer, but more preferably in the range of 4 to 12 wt % and even more preferably in the range of 6 to 9 wt %. In addition, the solvent should be sufficiently volatile to evaporate without undue agitation to prevent issues with the underlying enzyme, but not so volatile as to create problems with a spray or dipping process. In a preferred embodiment, the vinyl acetate component of the flux-limiting membrane includes about 40% vinyl acetate. In preferred embodiments, the flux-limiting membrane is deposited onto the enzyme domain to yield a domain thickness of from about 0.05 microns or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns, and more preferably still from about 5, 5.5 or 6 microns to about 6.5, 7, 7.5 or 8 microns. The flux-limiting membrane may be deposited onto the enzyme domain by spray coating or dip coating. In one aspect, the flux-limiting membrane is deposited on the enzyme domain by dip coating a solution of from about 1 wt. % to about 5 wt. % EVA polymer and from about 95 wt. % to about 99 wt. % solvent.

Other flux-limiting membranes may be used or combined, such as a membrane with both hydrophilic and hydrophobic regions to control the diffusion of analyte and optionally co-analyte to an analyte sensor. For example, a suitable membrane may include a hydrophobic polymer component such as a polyurethane, or polyetherurethaneurea, or blends of polymeric materials to form a single phase or multiphase membrane system.

The material that forms the basis of the hydrophobic matrix of the membrane can be any of those known in the art as appropriate for use as membranes in sensor devices and as having sufficient permeability to allow relevant compounds to pass through it, for example, to allow an oxygen molecule to pass through the membrane from the sample under examination in order to reach the active enzyme or electrochemical electrodes. For example, non-polyurethane type membranes such as vinyl polymers, polyethers, polyesters, polyamides, inorganic polymers such as polysiloxanes and polycarbosiloxanes, natural polymers such as cellulosic and protein based materials, and mixtures or combinations thereof may be used.

In one aspect, the flux-limiting layer comprises a hydrophobic-hydrophilic copolymer comprising a polylakylene oxide. For example, a hydrophobic-hydrophilic copolymer may comprise polyethylene oxide in a polyurethane polymer that includes about 20% hydrophilic polyethylene oxide. The polyethylene oxide portions of the copolymer are thermodynamically driven to separate from the hydrophobic portions (e.g., the urethane portions) of the copolymer and the hydrophobic polymer component. The 20% polyethylene oxide-based soft segment portion of the copolymer used to form the final blend affects the water pick-up and subsequent glucose permeability of the membrane.

### Bioactive Agents

In some alternative embodiments, a bioactive agent may be optionally incorporated into the above described sensor system, such that the bioactive diffuses out into the biological environment adjacent to at least one of the sensor components. For example, bioactive agents may be selected from anti-inflammatory agents, anti-fouling agents, anti-platelet agents, anti-coagulants, anti-proliferates, cytotoxic agents, anti-barrier cell compounds, or mixtures thereof.

### Flexible Substrate/Flex Circuit Sensor Assembly Adapted for Intravenous Insertion

In one aspect, an electrochemical analyte sensor assembly may be configured for an intravenous insertion to a vascular system of a subject. In order to accommodate the sensor within the confined space of a device suitable for intravenous insertion, the sensor assembly may comprise a flexible substrate. Preferably the flexible substrate is a flex circuit. The flex circuit may comprise at least one non-working electrode and at least one working electrode, the at least one working electrode having an electroactive surface capable of providing a detectable electrical output upon interaction with an electrochemically detectable species. A first layer may be placed in direct contact with a portion of the electroactive surface of the at least one non-working electrode and a second layer may be placed in direct contact with a portion of the electroactive surface of the one or more working electrodes. An enzyme layer comprising a hydrophilic polymer-enzyme composition capable of enzymatically interacting with an analyte so as to provide the electrochemically detectable species, may be placed in direct contact with and at least partially covering the second layer covering the working electrode. And a membrane may be placed such that it covers the hydrophilic polymeric layer, the first and second layers and at least a portion of the electroactive surfaces of the working and non-working electrodes. The flex circuit preferably is configured to electrically couple to a control unit. An example of a flex circuit and it construction is found in co-assigned U.S. Application Nos. 2007/0202672 and 2007/0200254, incorporated herein by reference in their entirety.

Medical devices adaptable to the sensor assembly as described above include, but are not limited to a central venous catheter (CVC), a pulmonary artery catheter (PAC), a probe for insertion through a CVC or PAC or through a peripheral IV catheter, a peripherally inserted catheter (PICC), Swan-Ganz catheter, an introducer or an attachment to a Venous Arterial blood Management Protection (VAMP) system. Any size/type of Central Venous Catheter (CVC) or intravenous devices may be used or adapted for use with the sensor assembly.

For the foregoing discussion, the implementation of the sensor or sensor assembly is disclosed as being placed within a catheter, however, other devices as described above are envisaged and incorporated in aspects of the invention. The sensor assembly will preferably be applied to the catheter so as to be flush with the OD of the catheter tubing. This may be accomplished, for example, by thermally deforming the OD of the tubing to provide a recess for the sensor. The sensor assembly may be bonded in place, and sealed with an adhesive (ie. urethane, 2-part epoxy, acrylic, etc.) that will resist bending/peeling, and adhere to the urethane CVC tubing, as well as the materials of the sensor. Small diameter electrical wires may be attached to the sensor assembly by soldering, resistance welding, or conductive epoxy, or by use of a prefabricated connector. These wires may travel from the proximal end of the sensor, through one of the catheter lumens, and then to the proximal end of the catheter. At this point, the wires may be soldered to an electrical connector.

The sensor assembly as disclosed herein can be added to a catheter in a variety of ways. For example, an opening may be provided in the catheter body and a sensor or sensor assembly may be mounted inside the lumen at the opening so that the sensor would have direct blood contact. In one aspect, the sensor or sensor assembly may be positioned proximal to all the infusion ports of the catheter. In this configuration, the sensor would be prevented from or minimized in measuring otherwise detectable infusate concentration instead of the blood concentration of the analyte. Another aspect, an attachment method may be an indentation on the outside of the catheter body and to secure the sensor inside the indentation. This may have the added advantage of partially isolating the sensor from the temperature effects of any added infusate. Each end of the recess may have a skived opening to 1) secure the distal end of the sensor and 2) allow the lumen to carry the sensor wires to the connector at the proximal end of the catheter.

Preferably, the location of the sensor assembly in the catheter will be proximal (upstream) of any infusion ports to prevent or minimize IV solutions from affecting analyte measurements. In one aspect, the sensor assembly may be about 2.0 mm or more proximal to any of the infusion ports of the catheter.

In another aspect, the sensor assembly may be configured such that flushing of the catheter (ie. saline solution) may be employed. This would allow the sensor assembly to be cleared of any material that may interfere with its function. The sensor assembly may also be configured such that the sensor can be contacted with a calibration solution and/or flushing solution while deployed such that the sensor can be calibrated while positioned in vivo in a subject. Methods for providing calibration and/or flushing solutions to the indwelling sensor include, for example, pumps or elevated IV bags.

### Sterilization of the Sensor or Sensor Assembly

Generally, the sensor or the sensor assembly as well as the device that the sensor is adapted to are sterilized before use in a subject. Sterilization may be achieved using radiation (e.g., electron beam or gamma radiation), ethylene oxide or flash-UV sterilization, or other means know in the art.

Disposable portions, if any, of the sensor, sensor assembly or devices adapted to receive and contain the sensor preferably will be sterilized, for example using e-beam or gamma radiation or other known methods. The fully assembled device or any of the disposable components may be packaged inside a sealed non-breathable container or pouch.

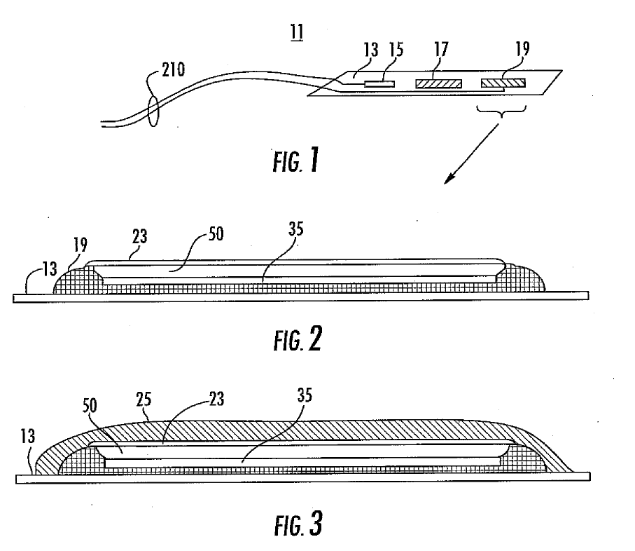

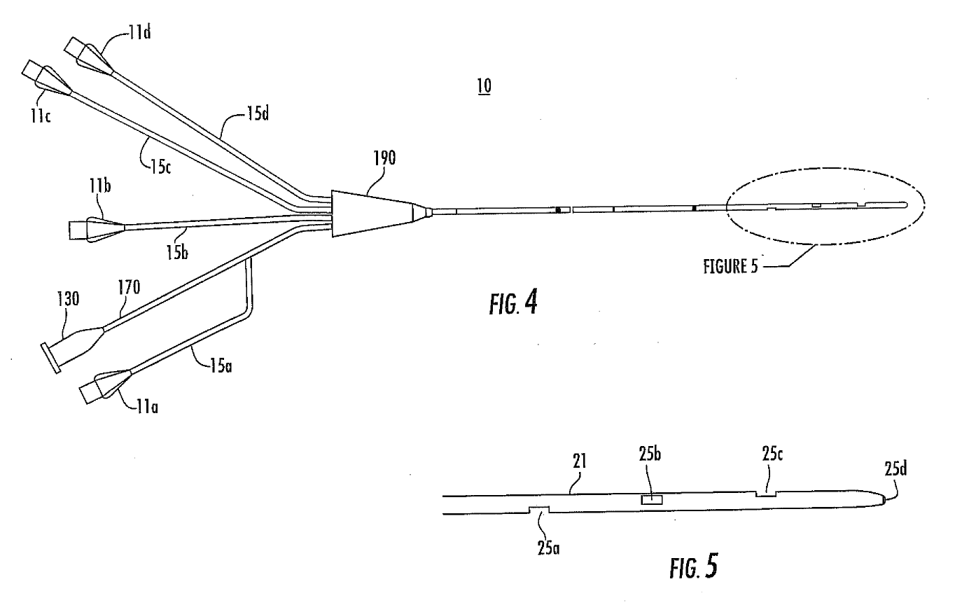

Referring now to the Figures, FIG. 1 is an amperometric sensor 11 in the form of a flex circuit that incorporates a sensor embodiment of the invention. The sensor or sensors 11 may be formed on a substrate 13 (e.g., a flex substrate, such as copper foil laminated with polyimide). One or more electrodes 15, 17, and 19 may be attached or bonded to a surface of the substrate 13. The sensor 11 is shown with a reference electrode 15, a counter electrode 17, and a working electrode 19. In another embodiment, one or more additional working electrodes may be included on the substrate 13. Electrical wires 210 may transmit power to the electrodes for sustaining an oxidation or reduction reaction, and may also carry signal currents to a detection circuit (not shown) indicative of a parameter being measured. The parameter being measured may be any analyte of interest that occurs in, or may be derived from, blood chemistry. In one embodiment, the analyte of interest may be hydrogen peroxide, formed from reaction of glucose with glucose oxidase, thus having a concentration that is proportional to blood glucose concentration.

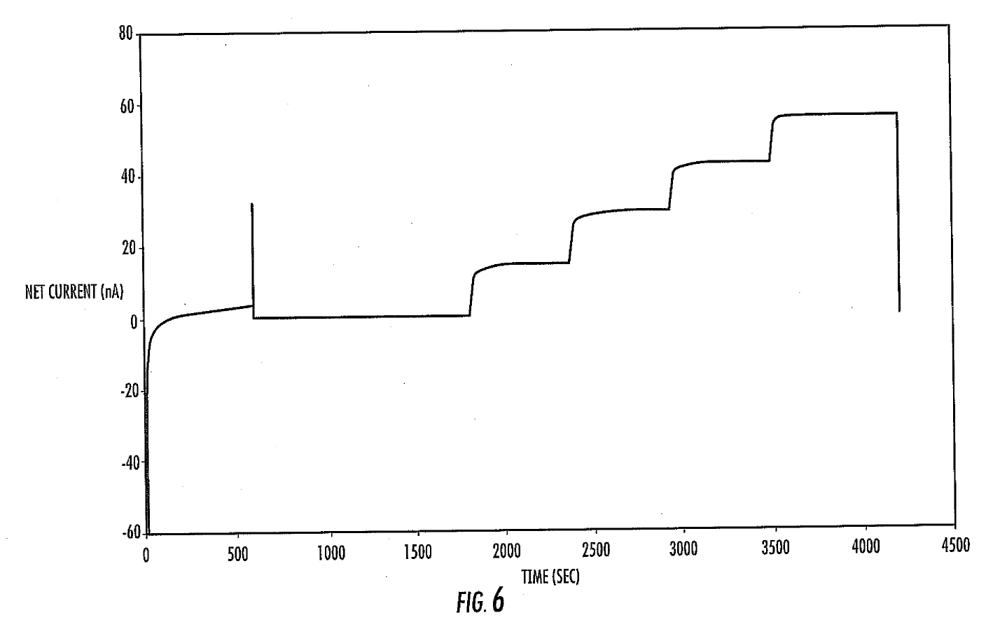

FIG. 2 depicts a cross-sectional side view of a portion of substrate 13 in the vicinity of working electrode 19 of an embodiment of the invention. Working electrode 19 may be at least partially coated with polyelectrolyte layer 35. Polyelectrolyte layer may be at least partially coated with interference layer 50. Interference layer 50 may be at least partially coated with an enzyme layer 23, the enzyme layer selected to chemically react when the sensor is exposed to certain reactants, for example, those found in the bloodstream. For example, in an embodiment for a glucose sensor, enzyme layer 23 may contain glucose oxidase, such as may be derived from Aspergillus niger (EC 1.1.3.4), type II or type VII.

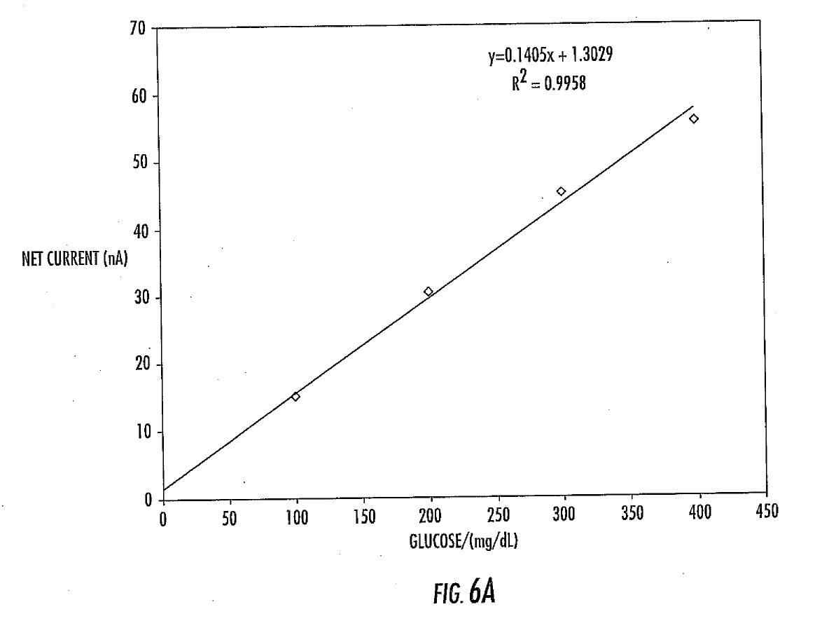

FIG. 3 shows a cross sectional side view of the working electrode site on the sensor substrate 13 further comprising membrane 25 covering enzyme layer 23, interference layer 50, polyelectrolyte layer 35 and at least a portion of electrode 19. Membrane 25 may selectively allow diffusion, from blood to the enzyme layer 23, of a blood component that reacts with the enzyme. In a glucose sensor embodiment, the membrane 25 passes an abundance of oxygen, and selectively limits glucose, to the enzyme layer 23. In addition, a membrane 25 that has adhesive properties may mechanically seal the enzyme layer 23 to the sub-layers and/or working electrode 19, and may also seal the working electrode 19 to the sensor substrate 13. It is herein disclosed that a membrane formed from an EVA polymer may serve as a flux limiter at the top of the electrode, but also serve as a sealant or encapsulant at the enzyme/electrode boundary and at the electrode/substrate boundary. An additional biocompatible layer (not shown), including a biocompatible anti-thrombotic substance such as heparin, may be added onto the membrane 25.

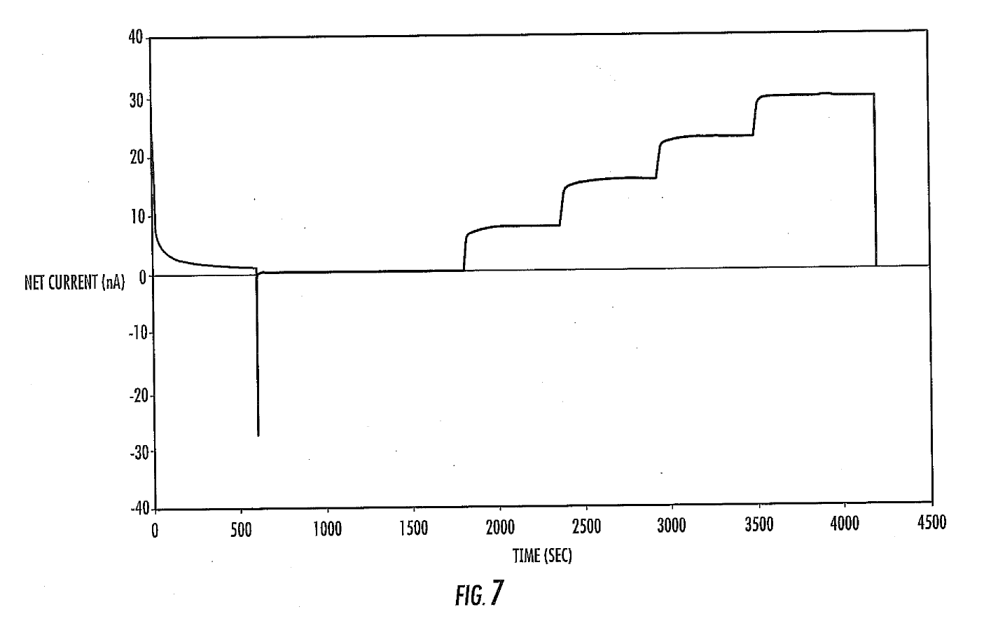

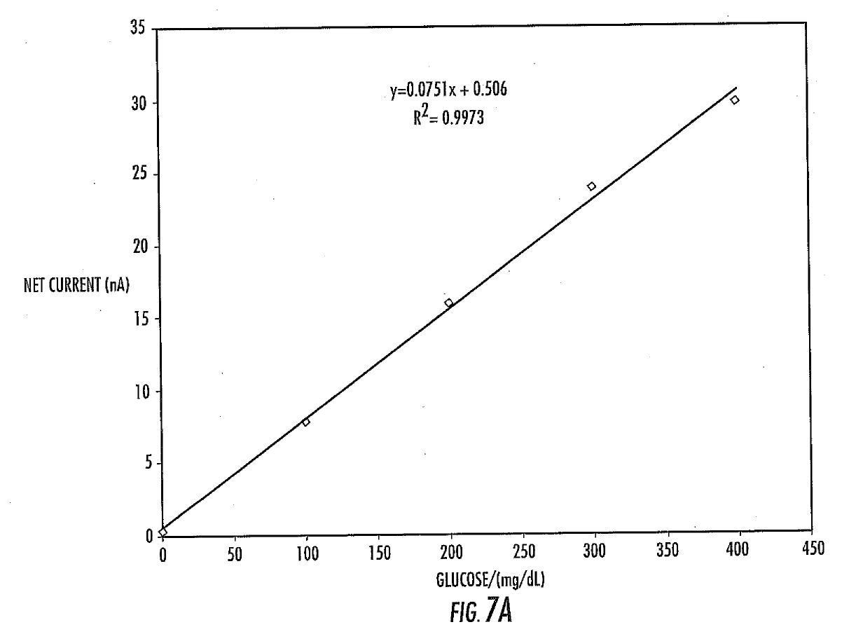

Referring now to FIGS. 4-5, aspects of the sensor adapted to a central line catheter with a sensor or sensor assembly are discussed as exemplary embodiments, without limitation to any particular intravenous device. FIG. 4 shows a sensor assembly within a multilumen catheter. The catheter assembly 10 may include multiple infusion ports 11a, 11b, 11c, 11d and one or more electrical connectors 130 at its most proximal end. A lumen 15a, 15b, 15c or 15d may connect each infusion port 11a, 11b, 11c, or 11d, respectively, to a junction 190. Similarly, the conduit 170 may connect an electrical connector 130 to the junction 190, and may terminate at junction 190, or at one of the lumens 15a-15d (as shown). Although the particular embodiment shown in FIG. 4 is a multilumen catheter having four lumens and one electrical connector, other embodiments having other combinations of lumens and connectors are possible within the scope of the invention, including a single lumen catheter, a catheter having multiple electrical connectors, etc. In another embodiment, one of the lumens and the electrical connector may be reserved for a probe or other sensor mounting device, or one of the lumens may be open at its proximal end and designated for insertion of the probe or sensor mounting device.

The distal end of the catheter assembly 10 is shown in greater detail in FIG. 5. At one or more intermediate locations along the distal end, the tube 21 may define one or more ports formed through its outer wall. These may include the intermediate ports 25a, 25b, and 25c, and an end port 25d that may be formed at the distal tip of tube 21. Each port 25a-25d may correspond respectively to one of the lumens 15a-15d. That is, each lumen may define an independent channel extending from one of the infusion ports 11a-11d to one of the tube ports 25a-25d. The sensor assembly may be presented to the sensing environment via positioning at one or more of the ports to provide contact with the medium to be analyzed.

Central line catheters may be known in the art and typically used in the Intensive Care Unit (ICU)/Emergency Room of a hospital to deliver medications through one or more lumens of the catheter to the patient (different lumens for different medications). A central line catheter is typically connected to an infusion device (e.g. infusion pump, IV drip, or syringe port) on one end and the other end inserted in one of the main arteries or veins near the patient's heart to deliver the medications. The infusion device delivers medications, such as, but not limited to, saline, drugs, vitamins, medication, proteins, peptides, insulin, neural transmitters, or the like, as needed to the patient. In alternative embodiments, the central line catheter may be used in any body space or vessel such as intraperitoneal areas, lymph glands, the subcutaneous, the lungs, the digestive tract, or the like and may determine the analyte or therapy in body fluids other than blood. The central line catheter may be a double lumen catheter. In one aspect of the present invention, an analyte sensor is built into one lumen of a central line catheter and is used for determining characteristic levels in the blood and/or bodily fluids of the user. However, it will be recognized that further embodiments of the invention may be used to determine the levels of other agents, characteristics, or compositions, such as hormones, cholesterol, medications, concentrations, viral loads (e.g., HIV), or the like. Therefore, although aspects of the present invention may be primarily described in the context of glucose sensors used in the treatment of diabetes/diabetic symptoms, the aspects of the invention may be applicable to a wide variety of patient treatment programs where a physiological characteristic is monitored in an ICU, including but not limited to blood gases, pH, temperature, and other analytes of interest in the vascular system.

In another aspect, a method of intravenously measuring an analyte in a subject is provided. The method comprises providing a catheter comprising the sensor assembly as described herein and introducing the catheter into the vascular system of a subject. The method further comprises measuring an analyte.

### EXAMPLES

It should be understood in the following examples that every percentage is a weight percentage unless otherwise noted. Further, it will be obvious to one of ordinary skill in the art that the following are offered for exemplary purposes and are in no way intended to limit the invention.

Preparation of sensors with a polyelectrolyte layer comprising polystyrene sulfonate-co-maleic acid copolymer(PSS-co-MA): A 3% PSS-co-MA solution was prepared by diluting a more concentrated PSS-co-MA with distilled water. An electrode utilizing carbon ink as an electroactive surface was treated with the PSS-co-MA solution. Wetting of the carbon ink was achieved by wiping the electroactive surface with the tip of a chemtip swab to apply a PSS-co-MA solution. The surface was then dried, producing a uniform film on the sensor surface. Three electrodes (a working, a blank, and a counter) were prepared in this fashion.

An interference layer was applied to the working and blank electrodes via treatment with a 4% CAB solution and dried. An enzyme layer deposited from a solution of about 1.5% glucose oxidase (GOX) with about 3.5% BSA and 40 μL of 12.5% gluteraldehyde was applied to the working electrode. A protein gel layer deposited from a solution of about 5% BSA solution and 10 μL of 12.5% gluteraldehyde was applied to the blank electrode. A membrane comprising 4% EVA was applied to the working, blank, and counter electrodes by dipping the electrodes in the EVA solution and allowing the electrodes to air dry.

Preparation of sensors with polyelectrolyte layers comprising heparin: A 1.3% solution of heparin in isopropyl alcohol (IPA) was applied to the electroactive surface of an electrode. Once the surface dried, an interference layer was applied by treating the electrode with a 0.2% CAB solution. The electrode was additionally treated with GOx as described in the preceding paragraph to produce the enzyme layer. The electrode was then dipped in a 6% EVA in xylenes solution at room temperature to apply a membrane.

Preparation of sensors without an interference layer: a PSS-co-MA solution was applied to an electroactive surface of an electrode. An enzyme layer was applied via treatment with GOx. The electrode was then dipped in a 6% EVA in xylenes solution at room temperature such that a membrane was applied to the electrode.

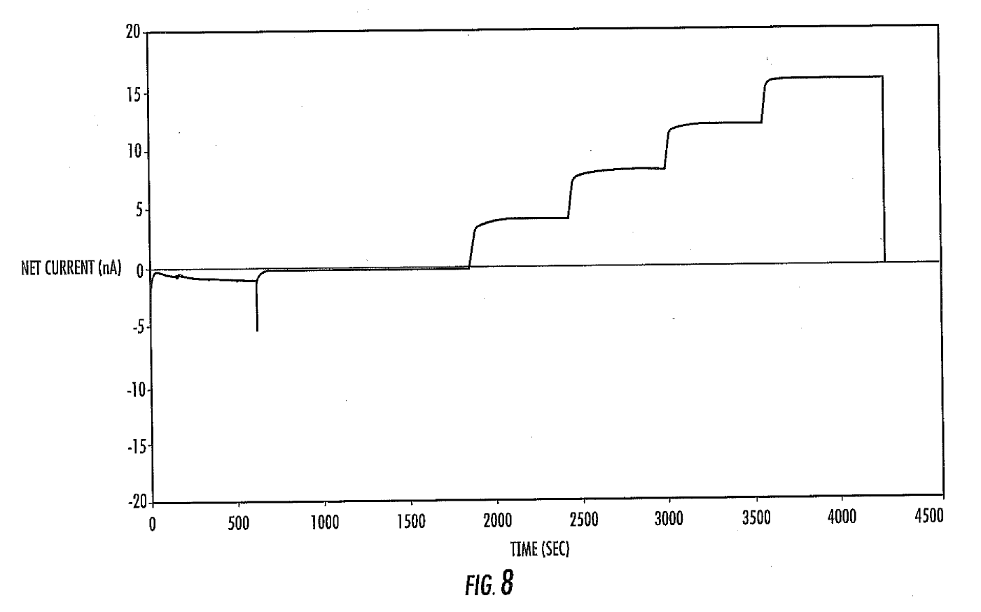

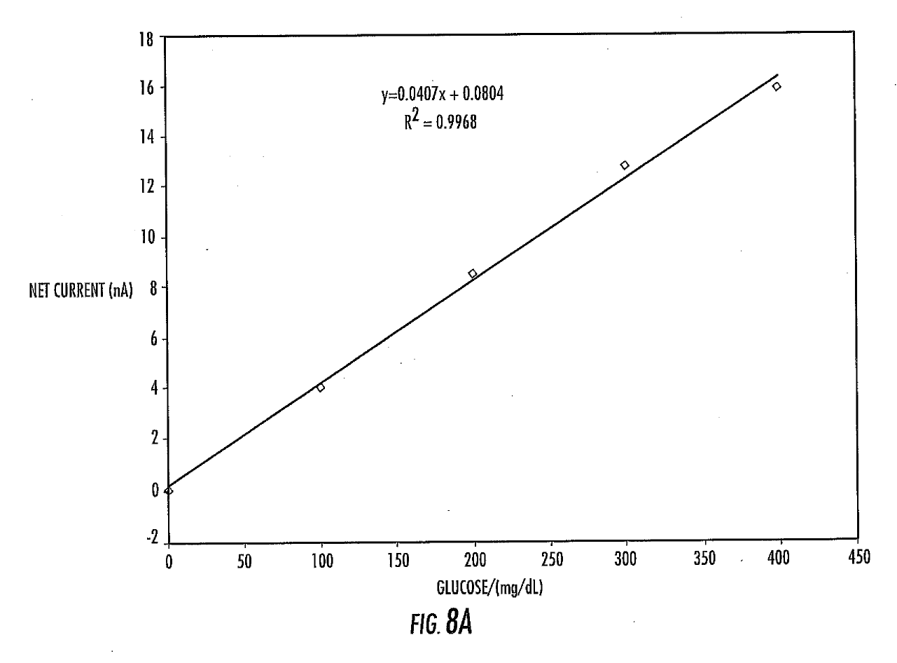

FIG. 6, 6A, 7, and 7A are graphical representations showing two exemplary sensors, respectively, each with the structure Electrode/PSS-co-MA/CAB/GOx/EVA. The sensors were subject to a glucose assay, where glucose concentration increased from 0 mg/dL to 400 mg/dL in 100 mg/dL increments. These sensors were run in for 30 minutes before the first glucose bolus. Current shown is the net current (working electrode-blank electrode) in nanoamperes (nA). The x-axis represents time in FIGS. 6 and 7 and glucose concentration in FIGS. 6A and 7A, while the y-axis in all 4 figures represents net current. FIGS. 6A and 7A demonstrate the sensors represented in FIGS. 6 and 7, respectively, comprising a PSS-co-MA polyelectrolyte layer provide a rapid and substantially linear glucose response.

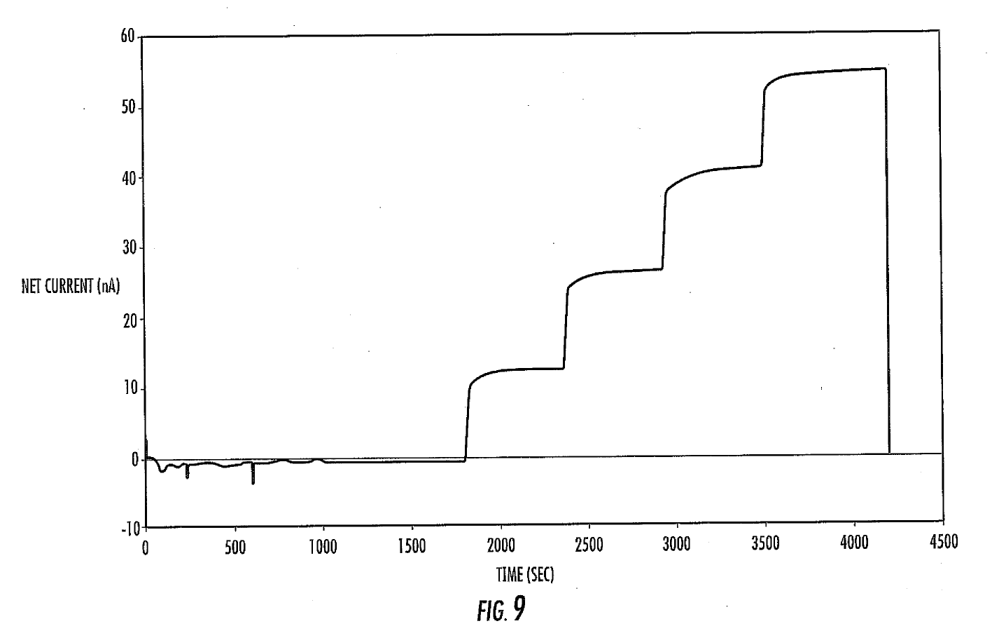

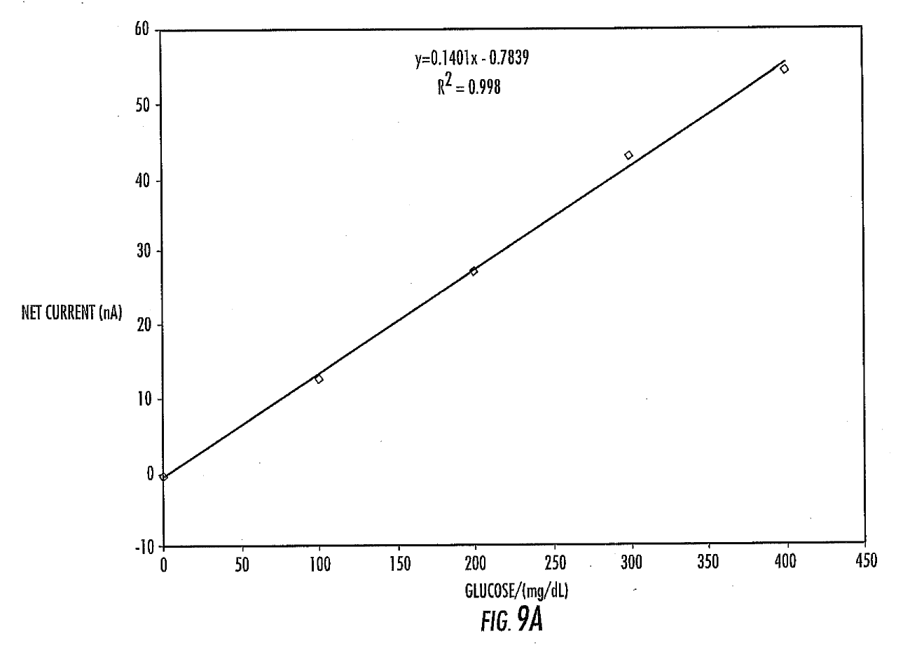

FIGS. 8, 8A, 9, and 9A are graphical representations showing two exemplary sensors, respectively, each with the structure Electrode/heparin benzalkonium polyelectrolyte/CAB/GOx/EVA. The sensors were subject to a glucose assay, where glucose concentration increased from 0 mg/dL to 400 mg/dL in 100 mg/dL increments. These sensors were run in for 30 minutes before the first glucose bolus. Current shown is the net current (working electrode-blank electrode) in nanoamperes (nA). The x-axis represents time in FIGS. 8 and 9 and glucose concentration in FIGS. 8A and 9A, while the y-axis in all 4 figures represents net current. FIGS. 8A and 9A demonstrates the sensors represented in FIGS. 8 and 9, respectively, comprising a benzalkonium heparin polyelectrolyte layer provide a rapid and substantially linear glucose response.

While some prior art interference layers have been known to create altered sensitivity of the sensor to glucose (e.g., variability and/or unreliability of sensors in manufacture), the instant glucose sensors, which were constructed with cellulose acetate butyrate interference layer in contact with at least a portion of a polyelectrolyte layer, and the polyelectrolyte layer in contact with at least a portion of an electroactive surface on an electrode, provide rapid and accurate glucose sensitivity and consistency. From the group of sensors represented in FIGS. 6-9, it may be approximated that at least in part, the polyelectrolyte layer provided for run-in times of about 11 minutes to about 15 minutes (using a criteria of less than 5 mg/dL glucose equivalent signal).

It has been shown that a sensor having an enzyme layer in contact with at least a portion of the polyelectrolyte layer as described herein, where the polyelectrolyte layer is in contact with the electroactive surface, produced accurate and consistent glucose sensitivity. It has further been shown that an interference layer is not required for these results but may be included to reduce false signals caused by known interferants.

It has been shown that a sensor with a polyelectrolyte layer between the electroactive surface and interference layer or between the electroactive surface and the enzyme layer provides rapid and accurate detectable output of the sensor, which reached a substantially constant value corresponding to the electrochemically detectable species.

Applicants believe the disclosure and data herein may be extrapolated to in vivo applications without undue experimentation by one of ordinary skill in the art.

Accordingly, sensors and methods have been provided for measuring an analyte in a subject, including a sensor assembly configured for adaption to a continuous glucose monitoring device or a catheter for insertion into a subject's vascular system having electronics unit electrically coupled to the sensor assembly.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

All numbers expressing quantities of ingredients, reaction conditions, and so forth used in the specification may be understood as being modified in all instances by the term “about.” Accordingly, unless indicated to the contrary, the numerical parameters set forth herein may be approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

What is claimed is:

1. An electrochemical analyte sensor comprising:
at least one non-reference electrode having an electroactive surface; and
a polyelectrolyte layer covering at least a portion of the electroactive surface of the at least one non-reference electrode.

2. The electrochemical analyte sensor of claim 1, wherein the polyelectrolyte layer comprises carboxylic acid functionality or sulfonate functionality.

3. The electrochemical analyte sensor of claim 2, wherein the polyelectrolyte layer comprises at least one of a polyacrylic acid, a polyalkylacrylic acid, a polystyrene sulfonate, a poly(sodium-4-styrene sulfonate), a poly(4-styrene sulfonic acid-co-maleic acid) sodium salt, a copolymer of polystyrene sulfonic acid, pharmaceutically acceptable salts thereof, or mixtures thereof.

4. The electrochemical analyte sensor of claim 1, wherein the polyelectrolyte layer comprises heparin, heparin salts, heparin benzalkonium salt, or mixtures thereof.

5. The electrochemical analyte sensor of claim 1, wherein the sensor further comprises an interference layer covering at least a portion of the polyelectrolyte layer.

6. The electrochemical analyte sensor of claim 5, wherein the interference layer comprises a cellulosic derivative selected from cellulose acetate, cellulose acetate butyrate, or mixtures thereof.

7. The electrochemical analyte sensor of claim 1, the sensor further comprising an enzyme layer at least partially covering the polyelectrolyte layer, wherein the enzyme layer optionally comprises a hydrophilic polymer.

8. The electrochemical analyte sensor of claim 7, wherein the hydrophilic polymer comprises a material selected from the group consisting of poly-N-vinylpyrrolidone, poly-N-vinyl-3-ethyl-2-pyrrolidone, poly-N-vinyl-4,5-dimethyl-2-pyrrolidone, polyvinylimidazole, poly-N—N-dimethylacrylamide, polyacrylamide, polyvinyl alcohol, polyethylene glycol, polyelectrolytes, and copolymers or blends thereof.

9. The electrochemical analyte sensor of claim 7, wherein the enzyme layer comprises an enzyme and a polyelectrolyte.

10. The electrochemical analyte sensor of claim 14, wherein the enzyme layer comprises glucose oxidase, poly-N-vinylpyrrolidone, and optionally an amount of crosslinking agent sufficient to immobilize the glucose oxidase.

11. The electrochemical analyte sensor of claim 1, further comprising a flux-limiting membrane covering the enzyme layer, the polyelectrolyte layer, and at least a portion of the electroactive surface.

12. The electrochemical analyte sensor of claim 11, wherein the flux-limiting membrane is selected from the group consisting of vinyl polymers, polysilicones, polyurethanes, and copolymers thereof.

13. The electrochemical analyte sensor of claim 11, wherein the flux-limiting membrane is poly(ethylene-co-vinylacetate).

14. The electrochemical analyte sensor of claim 1, further comprising at least one conductive ink electrode.

15. An electrochemical analyte sensor assembly comprising:
a flex circuit comprising at least one reference electrode and at least one working electrode, the at least one working electrode having an electroactive surface capable of providing a detectable electrical output upon interaction with an electrochemically detectable species;
a polyelectrolyte layer at least partially covering the electroactive surface of the working electrode, wherein the polyelectrolyte layer comprises carboxylic acid functionality or sulfonate functionality;
wherein the flex circuit is electrically configurable to a control unit capable of at least receiving the detectable electrical output.

16. The assembly of claim 15, wherein the polyelectrolyte layer comprises at least one of a polystyrene sulfonate or its pharmaceutically acceptable salts, poly(sodium-4-styrene sulfonate), poly(4-styrene sulfonic acid-co-maleic acid) sodium salt, heparin salts, heparin benzalkonium salt, pharmaceutically acceptable salts of polyacrylic acid or polyalkylacrylic acid, and mixtures thereof.

17. The assembly of claim 15 wherein the assembly further comprises an interference layer in contact with at least a portion of the polyelectrolyte layer.

18. The assembly of claim 17, wherein the interference layer comprises at least one of a cellulosic derivative, cellulose acetate, cellulose acetate butyrate, or mixtures thereof.

19. The assembly of claim 15, the assembly further comprising an enzyme layer at least partially covering the interference layer and the polyelectrolyte layer.

20. The assembly of claim 19, wherein the enzyme layer comprises a polyelectrolyte.

21. The assembly of claim 20, wherein the enzyme layer comprises glucose oxidase, poly-N-vinylpyrrolidone, and optionally an amount of crosslinking agent sufficient to immobilize the glucose oxidase.

22. The assembly of claim 15, wherein the sensor assembly further comprises a flux-limiting membrane at least partially covering the enzyme layer, the interference layer, and the polyelectrolyte layer.

23. The assembly of claim 22, wherein the flux-limiting membrane is selected from the group consisting of vinyl polymers, polysilicones, polyurethanes, and copolymers or blends thereof.

24. The assembly of claim 22, wherein the flux-limiting membrane is poly(ethylene-co-vinylacetate).

25. A method of intravenously measuring an analyte in a subject, the method comprising:
providing a catheter comprising the sensor of claim 1;
introducing the catheter into the vascular system of a subject; and
measuring an analyte.

26. The method of claim 25, further comprising contacting the sensor with a calibration solution while the catheter is in the vascular system of the subject.
